(19) United States
(12) Patent Application Publication
(10) Pub. No.: US 2018/0161437 A1
(43) Pub. Date: Jun. 14, 2018

(54) Foamed Medical devices with Additives

(71) Applicant: Udayan G. Patel, San Jose, CA (US)

(72) Inventor: Udayan G. Patel, San Jose, CA (US)

(73) Assignee: Udayan G. Patel, San Jose, CA (US)

(21) Appl. No.: 13/913,310

(22) Filed: Jun. 7, 2013

Related U.S. Application Data
(60) Provisional application No. 61/657,472, filed on Jun. 8, 2012.

(51) Int. Cl. A61K 47/30; A61F 2/82

(52) U.S. Cl. CPC A61K 47/30; A61F 2/82

(57) ABSTRACT

A medical device including an implantable medical prosthesis that can be reshaped into a scaffold to support the bodily tissues and bones. Additionally, the medical prosthesis relates to an intraluminal graft that would prevent the walls of the passageway from collapsing and includes a polymer containing additives. The base polymers and additives are biodegradable and/or bioabsorbale. The composite matrix of polymer and additives have lowered density through foaming having either closed cell foam or open cell foam or combination thereof. Alternately, the structural member of the device can be a hollow continuous tube or made of many hollow tubes (short) joined on ends thus making hollow longitudinal cells.

### CROSS-REFERENCE TO RELATED APPLICATIONS

This non-provisional application claims benefit of priority from U.S. provisional application No. 61/657,472, filed on Jun. 8, 2012, the contents of which are hereby incorporated by reference.

### BACKGROUND OF THE INVENTION

Most of the past research in the field of biodegradable implants has been directed toward orthopedic applications, for instance, in bioabsorbable screws and pins for internal fixation for bones. The device in fixation applications are usually passive and provide structural fixation. Bioabsobable polymers are also used in sutures where they provide the strength required to hold two tissue surfaces in close proximity. New and useful bioabsorbable medical devices are capable of being implanted inside narrow passages within the body. More recently, bioabsorbable polymers have been used in cardiovascular application such as in stents and heart valves.

Ideally, the material required for a balloon expandable prosthesis such as a vascular stent should be able to undergo deformation to attain low profile and then be able to expand and sustain strength at the treatment site. This property is typical in metals that are malleable. A typical stent made of stainless steel which is malleable.

The yield strength provides an indication of the materials ability to withstand initial deformation while the elongation provides information of the malleability of the material to deform. The ultimate strength provides information on the materials ability to withstand deformation forces before breaking.

Plastics in general do not have the tensile strength of metals and the elasticity of polymeric materials varies depending on other characteristics. A typically rigid polymer does not have high enough elongation to be malleable. On the other hand the polymer with extremely high elongation properties does not have adequate tensile strength. Unlike metals most polymers are viscoelastic and exhibit severe recoil after the deforming forces are removed.

The polymeric materials typically are either elastic or rigid with some materials having properties that fall between the extremes. Typically, a material with high elongation is soft and has low strength. On the other hand materials with high strength are rigid but, have low elongation. Such rigid materials are usually semi-crystalline. Rigid materials can be classified as brittle or ductile. Brittle materials tend to fracture easily upon impact or with very little elongation upon tensile deformation. Ductile materials tend to undergo certain elongation upon application of stress before complete fracture. Rigid materials can be quenched into mostly amorphous state to improve ductility however, they may lose their strength and especially ability to withstand cyclic stress.

Ideally, the stent material is required to have a high enough strength, sufficient enough elongation past its yield point and, not have any residual elasticity to cause recoil. Typical biodegradable materials listed in Table 1 do not meet the fullest requirements for intravascular stent. These materials have a very low elongation when functioning above their Tg. The functioning temperature of an implant typically being the body temperature (37° C.). Hence, a balloon expandable stent made from these materials crack upon slightest expansion in the body. On the other hand the materials that have their Tg below 37° C. have high enough elongation and the stents made from these materials can be expanded without cracking however, they do not have supporting structural strength.

Polymeric materials have visco-elastic properties. When stress is applied the plastic material initially undergoes elastic deformation before yielding into the non-elastic or viscous or plastic deformation region. The plastic deformation continues until the applied stress exceeds its elastic limits beyond which, the material fractures. When these materials are elongated within the elastic limits the material tends to regain their original shape. The time of recovery depends on the polymer structure of the material. Once the material is stretched beyond its elastic limits it undergoes permanent plastic deformation and will not be able to recover its original shape completely. During plastic deformation the polymer molecule chains undergoes extension and dislocations while maintaining some of the material integrity.

After the material has undergone plastic deformation the polymer chains become extended and are in a higher state of instability (low equilibrium state) as compared to that in its pre-deformation shape. Once plastically deformed the materials cannot recover to its pre-deformed shape however, the extended polymeric chains will tend to achieve the lowest energy state of equilibrium which, would be as in the pre-deformed state. The extent and time of the recovery depends on the environmental conditions that permit the movement of the polymer chains. The polymer chain movement is permitted above the glass transition temperature. The polymer chain movement is also permitted upon salvation by solvent molecules.

The extent of maximum elastic deformation and maximum plastic deformation of the material depends on the immediate environment such as temperature, fluid media, radiation, etc. Typically, temperature has the highest influence on characteristics of polymers. Above the glass transition temperature the polymer becomes more plastically deformable and the elastic recovery becomes limited but, with reduction in strength. Although, the deformed state can be frozen and, most of the strength is recovered. For structural application it is necessary to maintain the functional temperature of the plastic below its glass transition temperature. If in such applications the article is required to undergo an initial deformation while below its glass transition temperature then it is highly likely to form high stress points which can initiate cracks resulting in ultimate failure. On the other hand if the deformation is within the elastic limits then the final shape retention becomes difficult to control since, the material will tend to recoil back to its original shape.

Intravascular stents are typically made of metal which can be formed in situ. Traditionally, the stents are press fit over a balloon and delivered to the location of treatment within the vasculature, and thereafter expanded to appose against the blood vessel by inflating the balloon with pressurized fluid. Therefore, metals that are malleable as described above are most suitable for constructing such devices. More recently, stents made of biodegradable and/or bioabsorbable polymers are highly desired by clinicians because of the potential of relief from long term issues related to the biostable stents. However, the biodegradable or bioabsorbable materials are mostly rigid polymers at body temperature and they do not have the malleability of metals nor do they have adequate tensile elongation of stent metals. Hence, stent made from these bioabsorbable polymers tend to crack or facture upon expansion. To make these more malleable the glass transition temperature (Tg) is typically lowered by reducing molecular weight of the polymer or by blending with another polymer that has lower glass transition temperature (See Table 1). The blended materials can then be formed in situ however, due to lowered rigidity such a stent will not be able to maintain adequate support to the arterial wall.

TABLE 1
Some of the properties of biodegradable materials commonly
used or under investigation for application in implants
(see http://www.purac.com/Purac-
Biomaterials/EN/Products/Polymers-for-medical-devices.aspx).

| | Tg | UTS | Elongation |
|---|---|---|---|
| Materials | (C.) | (MPa) | % |
| PLLA | 60 | 65 | ~4 |
| PGA | 40 | 95 | ~2 |
| PDLLA | 55 | 40 | ~4 |
| PCL | −60 | 25 | >300 |
| PLGA (85 L/15 G) | 55 | 65 | ~4 |
| PLGA (82 L/18 G) | 50 | 60 | ~4 |
| PLGA (10 L/90 G) | 40 | 45 | ~4 |
| PDLGA (50 DL/50 G) | 45 | 45 | ~3 |
| PLDLA (80 L/20 DL) | 60 | 50 | ~4 |
| PLC (70 L/30 C) | 20 | 3 | >100 |

The bioabsorbable polymers are typically made from lactone based polyesters, polyorthoesters, polyanhydrides or based on para-dioxanone, trimethylene carbonate, caprolactone, and combinations thereof. Such materials breakdown over time by chemical hydrolysis in presence of water or enzymes or both and, get converted to low molecular metabolites. The rate of breakdown of such materials depends upon the speed with which these polymers get hydrolyzed. The rate of hydrolysis into metabolites depends upon many factors including the environment and the construction of the prosthesis. Certain bioabsorbable polymers swell up by bulk followed by slow hydrolysis while some other types of polymers undergo surface erosion.

Further, the metabolites generated as a result of hydrolysis of the bioabsorbable polymers have a slight acidic characteristics which cause local tissue inflammation. The inflammation has been observed in vascular systems where the vessels cause expansion or positive modeling of the vessel lumen. The extent of inflammation depends on the pH of the acid which in turn is dependant on the type and amount of acid produced during degradation. This inflammation is not typically observed in polymers that degrade by surface erosion such as polyorthoesters and polyanhydrides as the amount of acid released at a given time is small enough to not cause tissue inflammation. However, the metabolites generated by lactide based polymers are acidic in nature and may cause inflammatory reaction prior to their absorption.

Stents are made from materials ranging from metals to plastics. This invention is related to but not limited to plastic stents. It is one object of this present invention to reduce the recoil associated with elastic memory of the polymeric stent when expanded from its collapsed state to its final expanded state. It is also the objective to increase the rate of hydrolysis of the bioabsorbable polymeric stent. It is also the objective of the present invention to apply this invention to stents made of other materials such as metals to improve the expansion characteristics and reduce recoil of the stent. It is also the objective of this invention to promote endothelialization and cell growth through foam structure in the bulk of the prosthesis structural members and additionally on the surface. It is also the objective of this invention to provide extra capacity to load the bio absorbable prosthesis with therapeutic agents.

### SUMMARY OF THE INVENTION

The present invention is directed to but not limited to an endoluminal prosthesis. The present invention comprises of modified polymeric bulk configuration and material composition that can enhance properties of a vascular prosthesis and be absorbed or dissolved over time. Such materials can be used but not limited to, in construction of the endoluminal prosthesis that is required to be deformed in situ and then be able to provide mechanical strength until eroded and/or, degraded and/or, dissolved and/or, absorbed.

The modifications to the polymer are directed to suit the requirement of the implant by making increasing the pliability of the material while maintaining sufficient strength. This is achieved by addition of one or more property modifying materials or by creating a low density foam or combination thereof. The properties of the modified material are expected to meet or exceed the following but, not limited to the following criteria:

Ultimate Tensile Strength: >100 MPa

Yield Strength: >50 MPa

Elongation at break: >25%

Achieve above properties at body temperatures.

A rigid material can be bent within its elastic limits and would recoil back to its original shape. When the rigid material is bent beyond its elastic limits it plastically deforms during which the outer fibers at the bent location undergo extreme stress and may even tear from where cracks initiate. However, when a foam of the rigid material is bent the outer cells of the foam undergo extreme stress but any crack propagation is arrested. This is because the wall of the adjacent hollowed foamed cell would be in the path of the propagating crack. This is the characteristic of the foam. A foamed cell consists of a void surrounded completely or partially by the walls made of the base material. Further, by the mechanism of some of the cells breaking open during deformation the remaining underlying foam cells are protected from excessive stress. Further, the cells that remain intact at the deformation site also undergo some compression of the hollow void which, makes the overall structure more pliable than that of the base material. Thus, the ductility of a rigid material can be enhanced by creating foamed or cellular matrix.

Foam is formed by trapping gas bubbles within the solid. It consists of polydisperse gas bubbles separated by walls. When multiple gas bubbles are trapped completely within the confines of the material then it forms a closed cell foam. When the gas is not completely trapped on all sides by the material but it has open spaces to escape then the foam is an open celled foam. With foaming the material reduces its overall density. At low reduction in density the foam is usually closed cell. The foam typically becomes open cell when the density reduces below 50% of the density when in solid state. With lowering in density the material strength reduces but, not until a certain threshold reduction is reached. In some rigid materials the strength is shown to increase slightly for density reduction of up to 5%.

In this invention the rigid material with high Tg can be made to be more suitable for endovascular prosthesis such as a stent by making it into a foamed structure. The body of the stent is comprised of the foamed structural members forming a tubular structure. Each membrane comprises of foam with micro-cells. The density of the foamed member ranging from 99% of the base solid material density to about 25% that of the solid material. Ideally, the density of the foamed membrane range from 98% to 90% of the base polymer density. The foamed membrane may form a skin and the outer surfaces while the inside bulk of the polymer be foamed to the extent as stated above. On the other hand the outer surface is allowed to foam thus breaking the smooth surface and forming textured surface.

Potential base polymers are listed as but not limited to, polylactide, polyglycolide, polyparadioxanone, polycarbonate, polycaprolactone polyacetals, polycarbonates, polyanhydrides, polyorthoesters, polyglycolide, copolymers of glycolide, poly(glycolide-co-caprolactone), glycolide/L-lactide copolymers, lactide/trimethylene carbonate copolymers, glycolide/trimethylene carbonate copolymers, polylactides, stereo-copolymers of PLA, poly-L-lactide, poly-DL-lactide, L-lactide/DL-lactide copolymers, copolymers of PLA, lactide/tetramethylglycolide copolymers, lactide/.alpha.-valerolactone copolymers, lactide/.epsilon.-caprolactone copolymers, hyaluronic acid and its derivatives, polydepsipeptides, PLA/polyethylene oxide copolymers, unsymmetrical 3,6-substituted poly-1,4-dioxane-2,5-diones, carboxymethyl cellulose, poly-.beta.-hydroxybutyrate (PHBA), PHBA/bhydroxyvalerate copolymers (PHBA/HVA), poly-p-dioxanone (PDS), poly-a-valerlactone, poly-.epsilon.-caprolactone, methacrylate-N-vinyl-py-rrolidone copolymers, polyesteramides, polyesters of oxalic acid, polydihydropyranes, polyalkyl-2-cyanoacrylates, polyurethanes, polyvinylalcohol, polypeptides, poly-.beta.-malic acid (PMLA), poly-.beta.-alcanoic acids, polybutylene oxalate, polyethylene adipate, polyethylene carbonate, polybutylene carbonate, and other polyesters containing silyl ethers, acetals or ketals, and alginates, poly(glycolic acid), poly(l-lactic acid), poly(3-hydroxybutric acid), poly(dl-lactic acid), poly(d-lactic acid), poly(lactide/glycolide) copolymers, poly(hydroxyvalerate) or poly(hydroxyvalerate-co-hydroxybutyrate).

Within each foamed structural member of the stent the foam cells maybe discrete closed cell or continuous open cells. The wall between the foamed cells maybe thin or thick depending and the number and size of each cell. It will be appreciated by those in the field that the smaller the cells the thicker could be the wall whereas, the greater the number of cells the thinner the wall gets. The wall thickness may vary from nonexistent in case of open cell foam, to half the largest dimension of the device.

Additionally, each structural membrane maybe made up of a single foam cell thus, forming a structural member essentially comprising of a hollow polymeric tube. The stent could then be composed of several hollow tubes or of a single hollow tube of polymer. The wall thickness of the discretely hollow tubular members may vary from 49.5% of the maximum outer dimension of the tubular membrane to 5%. In this particular instance where the stent's structural members are composed of one or several hollow tubes the surface may not be textured. A plasticizing agent such as oils can be used as aids to allow ease of foaming. Therapeutic agents may be infused within the hollow tubular members.

The surface may also be made of different material than the base polymer thus, making the structural membranes made of multiple layers of different materials. Each layer may further be foamed to same extent or to different extents. Each layer of foamed material may be infused with same or different therapeutic agent.

In the current invention rigid materials with high Tg are made to enhance their malleability while still continuing to maintain their structural strength. Additives that soften the material and enhance the elongation are added to increase the pliability. These materials can be categorized as polymer with low Tg, low molecular weight waxes, and low molecular weight compounds. An example of a biodegradable polymer with low Tg is the polycaprolactone (PCL). PCL has Tg of minus 60° C. therefore it is extremely soft and pliable at body temperature however, it has very low strength as seen in table 1. In the current invention PCL is copolymerized or blended in small quantities between 3% to 15% to a rigid biodegradable plastic such as poly-1-lactic acid (PLLA). This gives a copolymer or a blend that has significantly increased elongation than that of pure PLLA at 37° C. The Tg and the mechanical properties of the new material can be maintained to the level that of the base polymer PLLA. A stent made of this material can thus be expanded at 37° C. without cracking and yet be able to maintain the structural integrity under the crushing forces bodily tissues.

In another embodiment low molecular weight oils can be blended with the biodegradable polymers to lower the Tg and make it more pliable. Examples of low molecular oils include triacetin, glycerine, vegetable oils, dioctylphthalate, etc. The amount of the oils added can be anywhere from 3% to 25% of the polymer.

An alternate method commonly used to improve the ductility is by addition processing aids such as of low molecular weight oils such as dioctyl phthalate. The oil particles when mixed with polymers acts as a partial solvent and also lower the polymer density but not to the extent that of foamed materials. As a result the polymer molecules have increased mobility and the glass transition temperature is lowered.

With lowering of Tg the strength of the material is also lowered. The strength of the polymeric materials is maintained and/or enhanced by addition of reinforcing materials such as fibers. The fiber embedded in the polymer provides reinforcement to the polymer and improves the mechanical properties. Any reduction in tensile property due to processing of the polymer would be compensated by incorporation of fibers. Fibers do not alter the chemical structure of the rigid material but provide the necessary strength. There are many types of fibers that can be categorized into biodegradable and biostable. Many biostable fibers are available and will not be mentioned here. The biodegradable and bioabsorbable fibers are derived from natural substances such as wood and plant husk. Natural fibers such as Kenuf, Hemp, or, Flax fibers are biodegradable fibers and have significantly high tensile strength. Fibers made out of the biodegradable polymers such as PLLA fibers are also very strong and can enhance the strength of the prosthesis provided these can be incorporated into the prosthesis without heating to its melt point. The polymeric material of the present invention can be composed of various formulations as given in table 2.

TABLE 2
Formulations of foam.

| | Formulation | | | | |
|---|---|---|---|---|---|
| | | | Process | Acid | Thera- |
| | Foam | Fibers | Aids | Neutralizer | peutics |
| | Range | | | | |
| | % density | volume | wt | molar | μgm/ |
| | reduction | % | % | % | sqmm |
| Possibly | 1 to 99% | 0 to 50 | 0 to 10 | 0-25 | 0-5 |
| Preferably | 1 to 25% | 0 to 35 | 0 to 7 | 0-10 | .01-3 |
| Ideally | 2 to 7% | 5 to 20 | 2 to 5 | .1-3 | .1-1 |

The fiber can enhance tensile properties even further if it has good adhesion to the polymer. The surface of the fibers can be made compatible or reactive by chemically altering the surface or simply subjecting the fibers to plasma of reactive gases.

The strength of the polymer can also be enhanced by addition of a cross linking agent. In case of PLLA a polyol may be added during polymerization. A cross-linked polymer is hard to process and formed into a prosthesis. Therefore, a partially cross linked polymer can be used.

The biodegradable materials undergo hydrolysis and breakdown into components that are slightly acidic. To neutralize these acids a mild base such as calcium carbonate or sodium bicarbonate can be added to the polymer. A biologically active agent or a compound can be added to the polymer during processing to improve biocompatibility as well as to improve the clinical outcome. Since, most polymers have very low density around 1 gm/cc these generally tend to be not visible under fluoroscopy or x-rays. A radiopaque element or combination of radiopaque elements can be added to the polymer to improve visibility under fluoroscopy. These radiopaque elements are the ones that have high density such as Os, Re, Pt, Au, Ir, W, etc.

The polymeric material can be infused with compounds that neutralize the acidic component of the hydrolysis.

These compounds could be but not limited to calcium carbonate or sodium bicarbonate.

The material can also contain a therapeutic agent for prevention of restenosis such as sirolimus or paclitaxel or derivatives thereof. Alternately, endothelial progenitor cell can be incorporated into the foamed struts to promote endothelialization. Finally, one or multiple types of proteins may be incorporated into the foamed matrix.

The stent can be made of multiple layers of different materials. Each layer consists of micro-cellular foam. The outer layer in contact with the vessel wall can be made of a softer bioabsorbable material that will impart less mechanical trauma than the inner rigid micro cellular foamed material. The inner rigid foam provides the necessary support for the vessel wall.

A therapeutic agent can be incorporated into the foamed structure. In a multi layered multi material foamed stent the therapeutic agent may be incorporated at different level of loading. Plasticizing oils can be added to allow ease of foaming. The amount of two of more biological agents on, in and/or used in conjunction with the medical device can be the same or different. In one non-limiting example, the medical device can be coated with and/or includes one or more biological agents such as, but not limited to, trapidil and/or trapidil derivatives, taxol, taxol derivatives (e.g., taxotere, baccatin, 10-deacetyltaxol, 7-xylosyl-10-deacetyltaxol, cephalomannine, 10-deacetyl-7-epitaxol, 7 epitaxol, 10-deacetylbaccatin III, 10-deacetylcephaolmannine, etc.), cytochalasin, cytochalasin derivatives (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin 0, cytochalasin P, cytochalasin Q, cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.), paclitaxel, paclitaxel derivatives, rapamycin, rapamycin derivatives, 5-Phenylmethimazole, 5-Phenylmethimazole derivatives, GM-CSF (granulo-cyte-macrophage colony-stimulating-factor), GM-CSF derivatives, or combinations thereof.

The foamed biodegradable stent material may contain fibers that impart additional strength to the stent. The fibers can be spun from biodegradable materials such as PLLA, PLGA, Polycaprolactone, etc., or those derived from natural sources such as Kenuf fibers, Flax fibers, hemp fibers, etc.

The structure of the foamed member or strut of the prosthesis can be a compound structure of open cell and closed cells as well as with or without a solid skin. A texture can be imparted to the surface or it may be a smooth outer surface. The foamed member may have a crystalline or amorphous matrix or could be semi-crystalline in nature.

Processing

The device can be fabricated by any of the conventional methods of making foamed parts such as but not limited to, injection molding with supercritical gas or extrusion of foamed tubing with supercritical gas and then carving the stent pattern from the tube. Addition of blowing agents during hot fabrication of the device is also one common method of making foam. The device can also be made by lamination process in which layers of material containing blowing agent is placed layers upon layer and then heated to activate the blowing agent to convert to foam.

### BRIEF DESCRIPTION OF THE DRAWINGS

FIG. 1 is the plan view of an embodiment of the stent configuration in an unexpanded condition.

FIG. 2 is the plan view of an embodiment of the stent configuration in an expanded configuration.

FIG. 3 is the plan view of a segment of the stent.

FIG. 4a is the end view of the segment in FIG. 3 disclosing one of the embodiments of this invention.

FIG. 4b is the end view of the segment in FIG. 3 disclosing another embodiment of this invention.

FIG. 4c is the end view of the segment in FIG. 3 disclosing yet another embodiment of this invention.

FIG. 4d is the end view of the segment in FIG. 3 disclosing yet another embodiment of this invention.

FIG. 4e is the end view of the segment in FIG. 3 disclosing yet another embodiment of this invention.

FIG. 4f is the end view of the segment of FIG. 3 disclosing another embodiment of this invention.

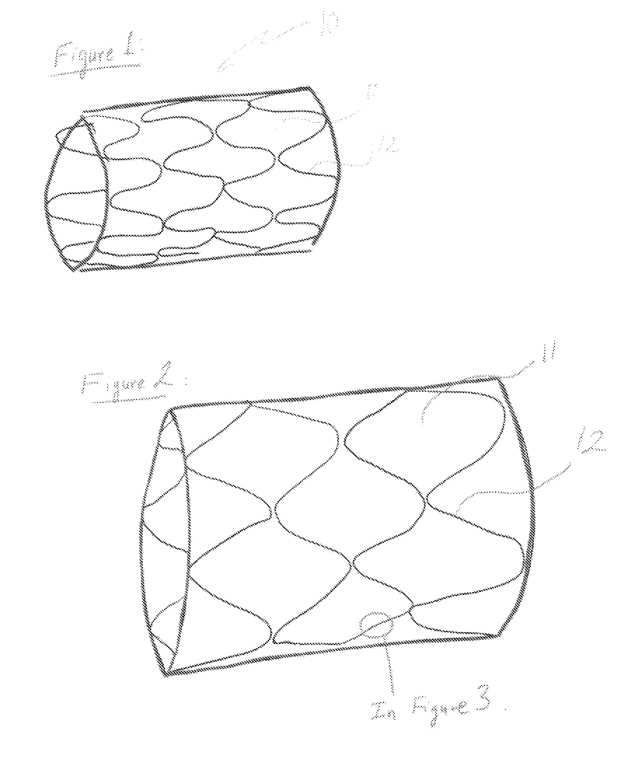

### DETAILED DESCRIPTION OF THE PREFERRED EMBODIMENTS

A preferred embodiment is a generally cylindrical endoluminal prosthesis 10 with micropores 11 constructed of members 12 made from materials whose density is lower than that of the base material. The preferred base material is but not limited, a polymeric material that will degrade or dissolve or be absorbed when by the surrounding fluids and tissues it is placed to support. The preferred endoluminal prosthesis is a stent. The lower density material that comprises each of the members 12 is obtained by creating a foam of the base material. The material is made into foam during the forming of the material into a stent or after the stent shape has been cut.

In FIG. 2 represents that the prosthesis 10 in an expanded state with the micropores 11 being enlarged as the individual members 12 are reoriented to accommodate the expanded shape. Upon expansion the members 12 that are generally aligned along the longitudinal axis of the stent 10 are now aligned at an angle to the longitudinal axis of the stent. During expansion a portion of the member undergoes bending deformation where some portions of the member are compressed and some portion is extended.

FIG. 3 shows a portion of the member 12 with an end 13 distinguishing the surface 15, also referred to as the skin, and exposing the inner foamed core 14 with lower density than the skin. The base material of the skin and the core could be the same of different than each other. The foamed core consists of alternating hollow cells 16 and solid matrix 17 of the polymer.

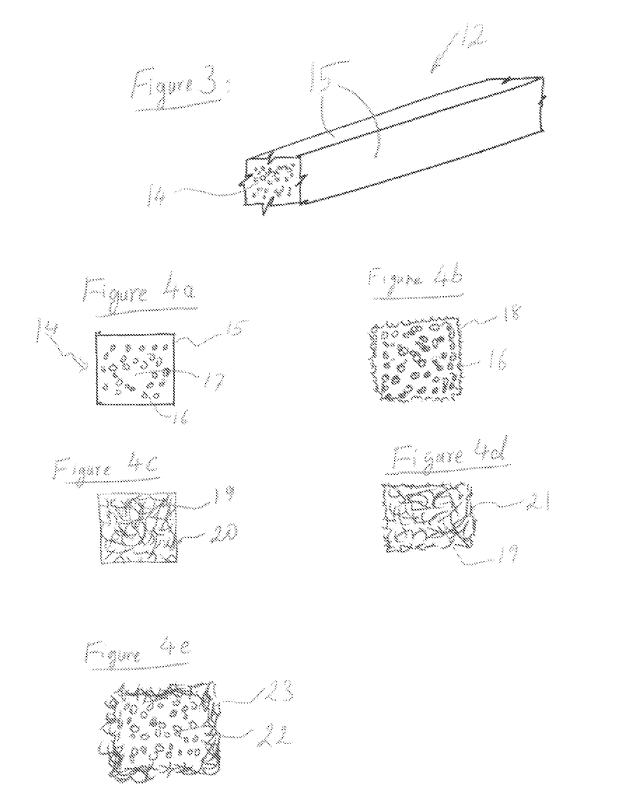

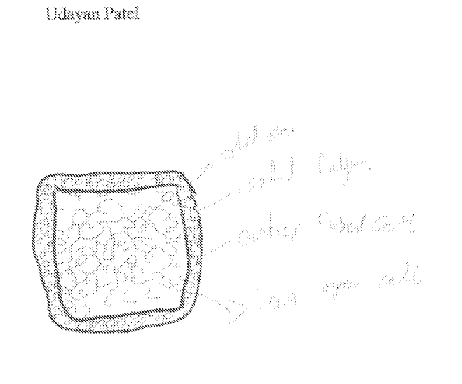

FIG. 4a is the cross section of the member 12 in which, the skin 15 and the core 14 are further defined. The skin 15 is solid and smooth. The foamed core consists of alternating hollow cells 16 and solid matrix 17 of the polymer. The cells are sized to give the foam density reduction of anywhere from 1% to 50%. FIG. 4b represents yet another embodiment showing the cross section of the member 12 in which, there is no separate solid skin layer but simply the closed cells of the core 16 are extended and exposed to the surface 18. This surface 18 is not a smooth uniform surface but, has geometry of the exposed hollowed void of the cell. FIG. 4c represents yet another embodiment showing the cross section of the member 12 in which, the foamed core consists of open cells 19 with a solid and smooth skin 20. The cells are sized to give the foam density reduction of anywhere from 1% to 50%. The open cells 19 are constructed such that each cell opens to one or more adjacent cell. FIG. 4d represents yet another embodiment showing the cross section of the member 12 in which, the foamed core consists of open cell 19 that extend to the outer surfaces 21. The outer surface is not smooth but, it is representative of the voids created by the exposed cell voids.

FIG. 4e represents yet another embodiment showing the cross section of the member 12 in which, the inner core 22 is made of closed cell foam while the outer skin is made of open celled foam 23. As shown in FIG. 4f, to those knowledgeable in the field it would also be possible where the core is made of open cells and the skin is made of closed cells. Further, the walls between the two types of cells could also be made of a solid skin as seen in FIG. 4f.

In another embodiment of this invention biodegradable fibers are integral part of the base polymer from which the members 12 are constructed. These fibers are, but not limited to natural fibers such as flax fibers, hemp fibers, bamboo fibers and, kenuf fibers. Additionally, fibers used by this invention are also made from but not limited to synthetic biodegradable polymer fibers.

In yet another embodiment of this invention the polymeric material is modified by addition of radiopaque material such as but not limited to high density elements and their compounds thereof. The high density materials are those that with densities greater than five grams per cubic centimeter.

In yet another embodiment of this invention the polymeric material is modified by addition of plasticizes or processing aids. These are but not limited to oils, stearates, phthalates, esters, adipates, triacetin, trimallitates, glycerine, polyols, oils, waxes, ethylene glycol, diethylene glycol, triethylene glycol, 2-ethylhexanol, isononyl alcohol, isodecyl alcohol, sorbitol, mannitol, PEG-500, PEG 1000 or PEG-2000 and low molecular weight polymers with glass transition temperatures lower than that of the base polymer. Additionally, modifiers are materials with glass transition temperature below that of the base polymer. The amounts in which these are added vary from zero to ten percent.

In yet another embodiment of this invention the polymeric material is modified by addition of a mild base. These are but limited to calcium carbonate and sodium bicarbonate.

In yet another embodiment of this invention the polymeric material is modified by addition of a biologically active agent or a compound that can improve biocompatibility as well as to improve the clinical outcome. These biological agent are but not limited to, sirolimus, sirolimus analogue, taxol and analogues, anticoagulants, ace inhibitors, alpha beta blockers.

The base polymer is summarized but not limited as indicated in Table 2 below:

TABLE 2
Formulations of foam.

| | Formulation | | | | |
|---|---|---|---|---|---|
| | | | Process | Acid | Thera- |
| | Foam | Fibers | Aids | Neutralizer | peutics |
| | Range | | | | |
| | % density | volume | wt | molar | μgm/ |
| | reduction | % | % | % | sqmm |
| Possibly | 1 to 99% | 0 to 50 | 0 to 10 | 0-25 | 0-5 |
| Preferably | 1 to 25% | 0 to 35 | 0 to 7 | 0-10 | .01-3 |
| Ideally | 2 to 7% | 5 to 20 | 2 to 5 | .1-3 | .1-1 |

While particular forms of the invention have been illustrated and described, it will be apparent that various modifications can be made without departing from the spirit and scope of the invention. It is contemplated that elements and structures from one embodiment may be combined or substituted with elements or structures from another embodiment.

1-27. (canceled)

28. An expandable tubular body, the expandable tubular body being a porous sleeve constructed of individual members wherein the pores in the sleeve are created between the individual members, wherein the individual members have outer skins defining outer boundaries of the individual members between which the pores in the sleeve are formed and wherein the individual members are at least partially comprised of a foamed material having a density ranging from 99% to 50% density of a base polymer from which the foamed material is formed and wherein the foamed material comprising the individual members is at least one of an open cell and a closed cell and includes biodegradable fibers ranging from 2 to 20 volume percent of the foamed material.

29. The expandable tubular body of claim 28, wherein the biodegradable fibers are selected from the group consisting of kenaf, bamboo, hemp, flax and combinations thereof.

30. The expandable tubular body of claim 28, wherein the biodegradable fibers are oriented either randomly, radially or longitudinally within the individual members of the expandable tubular body.

31. The expandable tubular body of claim 55, wherein the plasticizer is selected from the group consisting of triacetin, oils, stearates, phthalates, esters, adipates, trimallitates, glycerine, polyols, waxes and low molecular weight polymers with glass transition temperatures lower than that of the base polymer.

32. The expandable tubular body of claim 57, wherein said pH buffering agent is selected from the group consisting of sodium bicarbonate, calcium carbonate, calcium hydrozyapatite, tricalcium phosphate, carbonated calcium phosphates, magnesium hydroxide, amine monomer and lactate dehydrogenese.

33. The expandable tubular body of claim 28, wherein the expandable tubular body incorporates and controllably releases an effective amount of a sirolimus therapeutic agent.

34. The expandable tubular body of claim 58, wherein the radiopaque material is selected from the group consisting of platinum, iridium, rhenium and tungsten.

35. The expandable tubular body of claim 58, wherein the radiopaque material has a density greater than 8 gms/cc.

36. The expandable tubular body of claim 28, wherein at least a portion of the skin of the individual members of the expandable tubular body includes a non-foamed outer skin.

37. The expandable tubular body of claim 28, wherein at least a portion of the skin of the individual members of the expandable tubular body includes an open celled outer foamed skin.

38. The expandable tubular body of claim 28, wherein at least a portion of the skin of the individual members of the expandable tubular body includes a textured outer skin.

39. The expandable tubular body of claim 28, wherein at least a portion of the individual members of the expandable tubular body includes at least two different material layers that contain a therapeutic agent.

40. The expandable tubular body of claim 28, wherein the foamed material is comprised of a polylactide bioabsorbable material.

41. The expandable tubular body of claim 28, where the individual members of the expandable tubular body are formed as a device selected from the group consisting of a stent, a graft, a valve, a screw, a nail, a rod, a PFO device, a prosthetic device, a sheath, a guide wire, a balloon catheter, a hypotube, a catheter, a electrophysiology catheter and a cutting device.

42. The expandable tubular body of claim 28, wherein the skin of at least a portion of the individual members of the expandable tubular body are coated with a therapeutic agent.

43. The expandable tubular body of claim 28, wherein at least a portion of the individual members of the expandable tubular body includes a solid polymer.

44. The expandable tubular body of claim 28, wherein the expandable tubular body is included in a stent, suture, a balloon catheter, drug delivery implant, bone replacement, bone fixation device, dental implant, or ocular implant.

45. The expandable tubular body of claim 28, wherein at least a portion of the foamed members are comprised of biodegradable fibers up to 50 volume percent of the foamed members, and where the foamed members further include a plasticizer up to 10 weight percent of the foamed members, a pH buffer up to 25 molar percent of the foamed members and a therapeutic agent up to 5 μgm/mm² of the foamed member.

46. The expandable tubular body of claim 28, wherein said foamed material is comprised at least partially from biodegradable poly-1-lactic acid.

47. (canceled)

48. (canceled)

49. (canceled)

50. (canceled)

51. (canceled)

52. (canceled)

53. (canceled)

54. (canceled)

55. The expandable tubular body of claim 28 wherein at least a portion of the foamed material further includes a plasticizer comprising up to 10 weight percent of the foamed material.

56. The expandable tubular body of claim 28, wherein the biodegradable fibers comprises up to 50 volume percent of the foamed material.

57. The expandable tubular body of claim 28, wherein at least a portion of the foamed material further includes a pH buffer up to 25 mole percent of the foamed material.

58. The expandable tubular body of claim 28, wherein at least a portion of the foamed material further includes radiopaque material comprising up to 35 weight percent of the foamed material.

59. The expandable tubular body of claim 28, wherein at least a portion of the foamed material further includes a therapeutic agent comprising up to 5 μgm/mm² of the foamed material.

60. An expandable porous tubular body, the expandable porous tubular body constructed of individual members, wherein the individual members have external boundaries, wherein body pores are formed in the expandable porous tubular body by spaces between the external boundaries of the individual members, wherein the individual members are at least partially comprised of a foam formed within the external boundaries of the individual members, and wherein the foam includes foam pores of either or both an open or closed cell construction and further includes a plasticizer ranging from 2-10 percent of the foam by weight.

61. The expandable porous tubular body of claim 60, wherein the plasticizer is selected from the group consisting of triacetin, oils, stearates, phthalates, esters, adipates, trimallitates, glycerine, polyols, waxes and low molecular weight polymers with glass transition temperatures lower than that of the base polymer.

62. An expandable porous tubular body of claim 60 wherein the foam further includes biodegradable fibers ranging from 2-20 percent by weight of the foam.

63. The expandable porous tubular body of claim 62, wherein the biodegradable fibers are selected from the group consisting of kenaf, bamboo, hemp, flax and combinations thereof.

64. The expandable porous tubular body of claim 62, wherein the biodegradable fibers are oriented either randomly, radially or longitudinally within the individual members.

65. An expandable tubular body, the expandable tubular body being a porous sleeve constructed of individual members wherein the pores in the sleeve are created between the individual members, wherein the individual members have outer skins defining outer boundaries of the individual members between which the pores in the sleeve are formed;
wherein the individual members are at least partially comprised of a foamed material having a density ranging from 99% to 50% density of a base polymer from which the foamed material is formed and wherein the foamed material comprising the individual members is at least one of an open cell and a closed cell and includes biodegradable fibers ranging from 2 to 20 volume percent of the foamed material;
wherein the individual members further include up to 50 volume percent of kenaf fibers;
wherein at least a portion of the foamed material further includes triacetin plasticizer comprising up to 10 weight percent of the foamed material; and
wherein at least a portion of the foamed material further includes a pH buffer magnesium hydroxide up to 25 mole percent of the foamed material.
